(19) United States
(12) Patent Application Publication
(10) Pub. No.: US 2020/0168345 A1
(43) Pub. Date: May 28, 2020

(54) CROWDSOURCED PRIORITY FOR HEALTHCARE ETL

(71) Applicant: INTERNATIONAL BUSINESS MACHINES CORPORATION, Armonk, NY (US)

(72) Inventors: Paul R. Bastide, Boxford, MA (US); Shakil Khan, Highland Mills, NY (US); Ishwarya Rajendrababu, Hoboken, NJ (US); Yan S. Koyfman, Poughkeepsie, NY (US)

(21) Appl. No.: 16/202,863

(22) Filed: Nov. 28, 2018

(51) Int. Cl. G16H 80/00; G16H 70/20; H04W 4/02; G16H 40/20

(52) U.S. Cl. CPC G16H 80/00; G16H 70/20; G16H 10/60; G16H 40/20; H04W 4/023

(57) ABSTRACT

Embodiments of the present disclosure relate to prioritizing processing resources for a health care processing system. In various embodiments, a new message received by a health care processing system is detected. The message includes a plurality of parameters. A crowdsourced model is determined based on at least one of: geographical data from queries to the health care processing system, lock contention in the health care processing system, number of queries to the health care processing system, results of queries to the health care processing system frequently searched terms from websites, frequently occurring terms from websites, and trending topics from websites. A processing priority of the new message is determined based at least on the plurality of parameters and the crowdsourced model. The new message is assigned to a data processing queue based on the processing priority.

### BACKGROUND

Embodiments of the present disclosure relate to prioritizing processing resources for a health care processing system.

### BRIEF SUMMARY

According to embodiments of the present disclosure, systems, methods of and computer program products for prioritizing processing resources for a health care processing system are provided. In various embodiments, a new message received by a health care processing system is detected. The message includes a plurality of parameters. In various embodiments, a crowdsourced model is determined based on at least one of: geographical data from queries to the health care processing system, lock contention in the health care processing system, number of queries to the health care processing system, results of queries to the health care processing system, frequently searched terms from websites, frequently occurring terms from websites, and trending topics from websites. In various embodiments, a processing priority is determined for the new message based at least on the plurality of parameters and the crowdsourced model. In various embodiments, the new message is assigned to a data processing queue based on the processing priority.

In various embodiments, a system includes a computing node having a computer readable storage medium having program instructions embodied therewith. The program instructions are executable by a processor of the computing node to cause the processor to detect that a new message is received by a health care processing system. The message includes a plurality of parameters. In various embodiments, a crowdsourced model is determined based on at least one of: geographical data from queries to the health care processing system, lock contention in the health care processing system, number of queries to the health care processing system, results of queries to the health care processing system, frequently searched terms from websites, frequently occurring terms from websites, and trending topics from websites. In various embodiments, a processing priority is determined for the new message based at least on the plurality of parameters and the crowdsourced model. In various embodiments, the new message is assigned to a data processing queue based on the processing priority.

In various embodiments, a computer program product is provided for prioritizing processing resources for a health care processing system. The computer program product includes a computer readable storage medium having program instructions embodied therewith. The program instructions are executable by a processor to cause the processor to detect that a new message is received by a health care processing system. The message includes a plurality of parameters. In various embodiments, a crowdsourced model is determined based on at least one of: geographical data from queries to the health care processing system, lock contention in the health care processing system, number of queries to the health care processing system, results of queries to the health care processing system, frequently searched terms from websites, frequently occurring terms from websites, and trending topics from websites. In various embodiments, a processing priority is determined for the new message based at least on the plurality of parameters and the crowdsourced model. In various embodiments, the new message is assigned to a data processing queue based on the processing priority.

### BRIEF DESCRIPTION OF THE SEVERAL VIEWS OF THE DRAWINGS

FIG. 1 illustrates an exemplary diagram of an Extract-Transform-Load (“ETL”) data pipeline according to various embodiments of the present disclosure.

FIG. 2 illustrates an exemplary diagram of an ETL data pipeline according to various embodiments of the present disclosure.

FIG. 3 illustrates an exemplary system for prioritizing healthcare resources according to various embodiments of the present disclosure.

FIG. 4 illustrates a flow chart illustrating an exemplary method for prioritizing processing resources for a health care processing system according to various embodiments of the present disclosure.

FIG. 5 depicts an exemplary computing node according to various embodiments of the present disclosure.

### DETAILED DESCRIPTION

In big data applications, data generally flows from various data sources (also called data streams) into a data reservoir where the data is eventually processed and stored in a data warehouse and/or data mart for consumption by various applications, such as business intelligence tools.

Data reservoirs enable all forms of customer specific data to be stored in a uniform, single large storage repository for access by a data processing engine. Data reservoirs may be used for multi-dimensional analytics to discover optimal business outcomes. Data reservoirs may be single-tenant, where the data is stored and owned by a single entity, or multi-tenant, where data is stored and owned by multiple entities. Multi-tenant data reservoirs are quickly becoming a pattern in industry, and these data reservoirs isolate specific tenant data from all other tenants. Multi-tenant data reservoirs may maximize storage use of a database and provide uniform security and decryption of data. In various embodiments, the data reservoir may include certain predetermined permissions, such as, for example, read-only access to one or more preselected systems and read/write access to other preselected systems.

A data warehouse is a central repository of integrated data from one or more disparate data sources. They store current and historical data in one single place that are used for creating analytical reports for workers throughout the enterprise. In various embodiments, the data warehouse may include certain predetermined permissions, such as, for example, read-only access to one or more preselected systems and read/write access to other preselected systems.

Various cloud-based health record systems providers offer multi-tenant healthcare solutions where Electronic Health Records (“EHR”), Protected Healthcare Information (“PHI”), and/or patient medical data are stored together from multiple vendors, customers, and/or organizations in a single database and/or logical processing engine. Exemplary vendors may include, for example, hospitals, insurance providers, pharmacies, health care providers, etc. Data elements (which may be, e.g., structured and/or unstructured data) from various sources may be processed using an Extraction-Transformation-Load (“ETL”) system to thereby load the data into the data reservoir and/or a datamart for consumption by a specific business group. As a new data element (e.g., HL7 message, ADT message) is received, a pipeline may execute stages to complete the ETL process.

ETL is normally a continuous, ongoing process with a well-defined workflow. ETL first extracts data from structured or unstructured data sources. Then, data is cleansed, enriched, transformed, and stored either back in the data reservoir or in a data warehouse (or datamart within the data warehouse). Each incoming message (1 Kilobyte, 1 Gigabyte) may require a period of time (e.g., several seconds) to fully process through the ETL system. As new messages are queued for processing by the ETL system, ETL systems generally sequentially process the new messages thereby uploading the processed data/message to a datamart. In various embodiments, as an intermediate step, the ETL system may spread the load out across many systems, which execute the ETL.

However, some data messages may be more important than others in view of certain situations, and thus should be prioritized for processing by the ETL system. With the importance of real-time access to healthcare data, there is a need to optimize the processing of the data messages by ETL systems.

The systems, methods, and computer program products of the present disclosure relate to prioritizing processing resources for a health care processing system. In particular, incoming data messages to an ETL system are prioritized for processing based on a predetermined, crowdsourced model. With the rise of crowdsourcing, decisions are being supported by a diverse set of task workers, who each execute a nominal unit-of-work to generate a result. The result is statistically correlated with all other results of a similar nominal unit-of-work to confirm the result. The use of crowdsourcing techniques is untapped for healthcare decisions and Healthcare ETL.

In various embodiments, a new data message may be detected as being received by a health care processing system. The health care processing system may be an electronic health record (“EHR”) system comprising patient medical data. The new data message may originate from any suitable source, (e.g., a spreadsheet, a camera, a laptop, mobile phone, a text document, a relational database, NoSQL database) and may include structured or unstructured data. In various embodiments, the new data message may include metadata. In various embodiments, the metadata may include TCP/IP information, such as IP address, region, and/or total hops. In various embodiments, the metadata may include location, such as GPS location. In various embodiments, the metadata may include a message type, such as HL7, ORU, ADT, and/or FHIR. In various embodiments, the metadata may include a generation time. In various embodiments, the metadata may include a reception time. In various embodiments, the message may include content, such as {“condition”:“flu like symptoms, ex Diffuse otitis externa”}, a patient identifier (e.g., SID: 123456), a location, and/or a total time.

In various embodiments, a crowdsourced model may be determined from a number of sources. In various embodiments, the crowdsourced model may be based on geographical data from queries to the health care processing system (e.g., hotspots), lock contention in the health care processing system, number of queries to the health care processing system, results of queries to the health care processing system, frequently searched terms from websites (e.g., search engines, healthcare websites, and/or social media), frequently occurring terms from websites (e.g., news, healthcare websites, and/or social media), trending topics from websites (e.g., news, healthcare websites, and/or social media), and/or metadata/data-definitions of data collections in the data reservoir (e.g., parquet, Avro, relational).

In various embodiments, the crowdsourced model may include any suitable number and types of task workers. In various embodiments, the set of task workers for the crowdsourced model may include: 1.) through logging, the result of queries done by users of a data reservoir; 2.) the search terms from healthcare websites/search engines/specific solutions, through tracing of the websites; 3.) trending topics in Social media, extracting social data via Gnip; 4.) trending topics or swarming locations from reviews (location info can be extracted from mobile applications such as, for example, Facebook, Twitter, Yelp, Foursquare, and/or Swarm); 5.) the most frequently mentioned term in the health care review boards, doctor review websites, search engines (Google, Yahoo, Bing), and/or trend tracking systems (e.g., Google trends).

In various embodiments, a processing priority may be determined of the new data message based on the metadata and the crowdsourced model. In various embodiments, the processing priority may be determined by determining a weight of the new data message by, for example, comparing the extracted data and/or metadata in the message to the crowdsourced model. In various embodiments, the weight may be a value between 0.00 and 1.00. In various embodiments, the weight is used to assign the new data message to a low priority processing queue or a high priority processing queue. In various embodiments, a weight of 1.00 represents a high priority message while a weight of 0.00 represents a low priority message. In various embodiments, a range from 0.51 to 1.00 represents a high priority new data message. In various embodiments, a range from 0.00 to 0.50 represents a high priority new data message.

In various embodiments, where a first new data message having a first weight (e.g., 0.75) is placed in the high priority processing queue and a second new data message having a higher weight (e.g., 0.99) is also placed in the high priority processing queue, the second new data message may be placed ahead of the first new data message. In another example, where a first new data message having a first weight (e.g., 0.99) is placed in the high priority processing queue and a second new data message having a lower weight (e.g., 0.75) is also placed in the high priority processing queue, the second new data message may be placed behind of the first new data message. The same principle may be applied to the low priority processing queue.

In various embodiments, the crowdsourced model may be updated constantly in real-time as new data is received or at predetermined times (e.g., daily, weekly, monthly, etc.). In various embodiments, the crowdsourced model may be built as a historic model from a predetermined amount of time (e.g., past hour, past day, past week, past month, past year, or a normalized time unit). In various embodiments, the model may be based on a specific Time-to-Live or Time-to-Incubate for a disease.

In various embodiments, assigning the new message to a data processing queue based on the processing priority. In various embodiments, the data processing queue may be a low priority processing queue. In various embodiments, the data processing queue may be a high priority processing queue that processes new data messages before any new data messages that are in the low priority processing queue.

As an example, when new data message appears in the ETL pipeline input, the input processor solicits a crowdsourcing model to identify necessary and sufficient data features to suggest helpful tips such as (“There is a Zika break out in Miami, Fla.”) in response to message posted in the personal social network such “Visiting Miami, Fla. Tomorrow.”

In another example, an incoming data message to the ETL pipeline may include the location information as “Florida.” The crowdsourced model may include the following aggregated search terms from the sources: 1.) “Flu” is part of the observation result that is queried against the data reservoir where the location is “Florida”; 2.) “Zika” is the most frequently searched term in a health care website like “WebMD” at “Florida”; 3.) “Zika” is the most frequently searched term via “Google” at “Florida”; 4.) “Zika” is the most frequently analyzed term in some specific solution such as “Watson EMRA” where the location is “Florida”; 5.) “Flu” is the most frequently mentioned term in the reviews in “Yelp/Foursquare” at “Florida”; 6.) “Hepatitis” is the most frequently mentioned term in the State's Department of Health website, health care review boards, doctor review websites such as “Zocdoc” where the location is “Florida”; 7.) “Lyme disease” is the most frequently mentioned term in the social media such as “Twitter/Facebook” at “Florida.” The crowdsourced model may analyze the above results for the location “Florida” and assign the following exemplary confidence values for each of the above terms: {“Zika”, 10}, {“Flu”, 7}, {“Hepatitis”, 3}, and {“Lyme disease”, 3}.

In the above example, a new data message #1, which has “Florida” as the location info, is received and is determined by the system as more likely to have “Zika” as the observation result, hence it is assigned a higher priority during the ETL processing. The new data message #1 which has “Florida” as the location info and “Zika” as the observation result can be processed in a high priority queue ahead of other messages in a lower priority queue. Since “Zika” is the crowd sourced term with a higher priority, it is likely to receive more messages with location as “Florida” and these messages can be intelligently queued to achieve optimal/higher performance using priority queues and/or a cache mechanism.

In various embodiments, the crowdsourced model may be developed by retrieving the crowdsourced search terms from one or more users related to the location in the message.

In various embodiments, the weighted processing may trigger a priority execution of prior unprocessed message for a specific patient—e.g., an admit message is received and determined to be of low value. Subsequently, a diagnosis message is received and determined to be of high value, and the admit message is then processed prior to the diagnosis message for the patient.

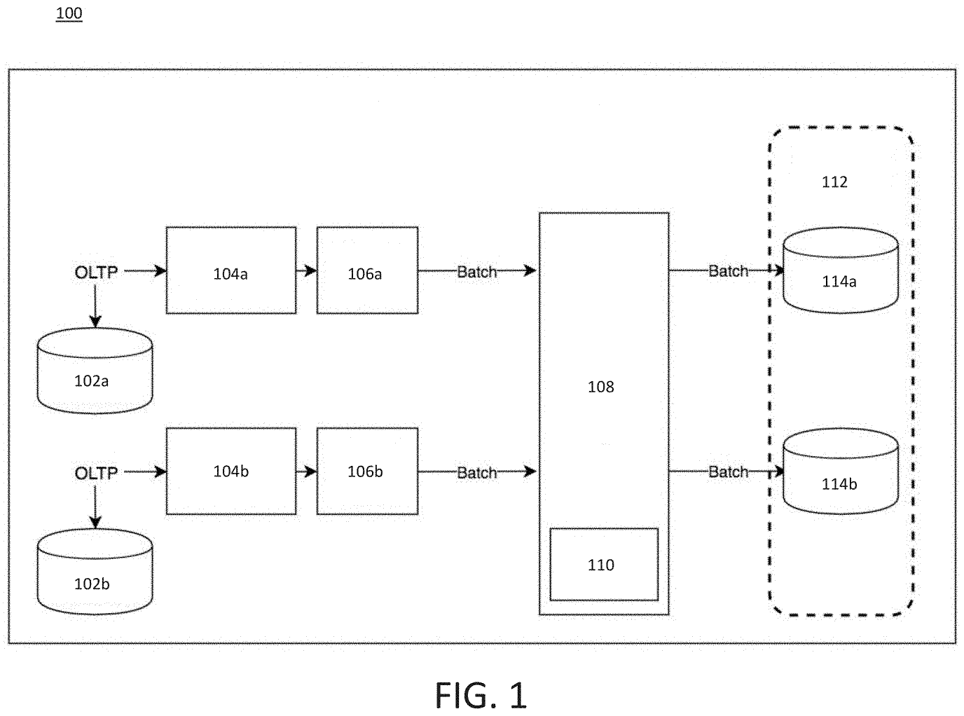

FIG. 1 illustrates an exemplary diagram of an Extract-Transform-Load (“ETL”) data pipeline 100 according to various embodiments of the present disclosure. As shown in FIG. 1, the data pipeline includes a first OLTP (On-line Transaction Processing) database 102a and a second OLTP database 102b. An OLTP process may be characterized by a large number of short on-line transactions (INSERT, UPDATE, DELETE) and may include very fast query processing, maintaining data integrity in multi-access environments, and an effectiveness measured by number of transactions per second. In an OLTP database, there may be detailed and current data, and schema used to store transactional databases is the entity model (such as, for example, 3NF).

In various embodiments, data from the first OLTP database 102a may flow into a first OLTP application 104a and data from the second OLTP database 102b may flow into a second OLTP application 104b. In various embodiments, the ETL data pipeline 100 may include change detection subsystems 106a, 106b that each receive respective data from the OLTP applications 104a, 104b. In various embodiments, the change detection subsystems 106a, 106b may be designed to detect changes in the operational data and to selectively forward new and changed data to the next stage of the ETL pipeline 100. Change detection may be especially critical when operational data is large because, for example, re-loading all of the operational data into the data warehouse every day would take a vast amount of processing resources. In various embodiments, the change detection subsystem 106a, 106b may be applied early on in the extract stage to minimize the size of data transfers; capture all of the changes (deletions, insertions and updates) using audit columns, database log scraping, timed extracts, diff compare, etc.; add flags to changed data identifying the reason for the change; and provide audit metadata (for compliance purposes).

In various embodiments, the ETL pipeline 100 includes an ETL system 108 configured to receive data in batches from the change detection subsystems 106a, 106b for processing. In various embodiments, the ETL system 108 extracts data from source systems (e.g., SAP, ERP, other operational systems) and data from the different source systems may be converted into one consolidated data warehouse format, which is ready for transformation processing. In various embodiments, the ETL system 108 transforms the received data by, for example, applying business rules (e.g., calculating new measures and dimensions), cleaning (e.g., mapping NULL to 0 or “Male” to “M” and “Female” to “F” etc.), filtering (e.g., selecting only certain columns to load), splitting a column into multiple columns and vice versa, joining together data from multiple sources (e.g., lookup, merge), transposing rows and columns, and/or applying any kind of simple or complex data validation as is known in the art (e.g., if the first 3 columns in a row are empty then reject the row from processing). In various embodiments, the ETL system 108 loads the data into a data warehouse or data repository.

In various embodiments, the ETL system 108 may include a dimension manager 110. In various embodiments, the dimension manager may be a centralized authority that prepares and publishes conformed dimensions to the data warehouse community. In various embodiments, a conformed dimension is by necessity a centrally managed resource where each conformed dimension must have a single, consistent source. In various embodiments, the responsibility of the dimension manager 110 is to administer and publish the conformed dimension(s) for which it has responsibility. In various embodiments, there may be multiple dimension managers in an organization's ETL pipeline. In various embodiments, the dimension manager's responsibilities may include the following ETL processing: implement the common descriptive labels agreed to by the data stewards and stakeholders during the dimension design; add new rows to the conformed dimension for new source data, generating new surrogate keys; add new rows for Type 2 changes to existing dimension entries (true physical changes at a point in time), generating new surrogate keys; modify rows in place for Type 1 changes (overwrites) and Type 3 changes (alternate realities), without changing the surrogate keys; update the version number of the dimension if any Type 1 or Type 3 changes are made; and/or replicate the revised dimension simultaneously to all fact table providers.

In various embodiments, the ETL pipeline 100 includes a logical data warehouse 112 to store the processed data. In various embodiments, the data may be passed from the ETL system 108 to the data warehouse 112 in batches. In various embodiments, the data warehouse may include one or more datamarts 114a, 114b where specific data is stored for consumption by, e.g., a specific business group within an organization or a specific external consumer.

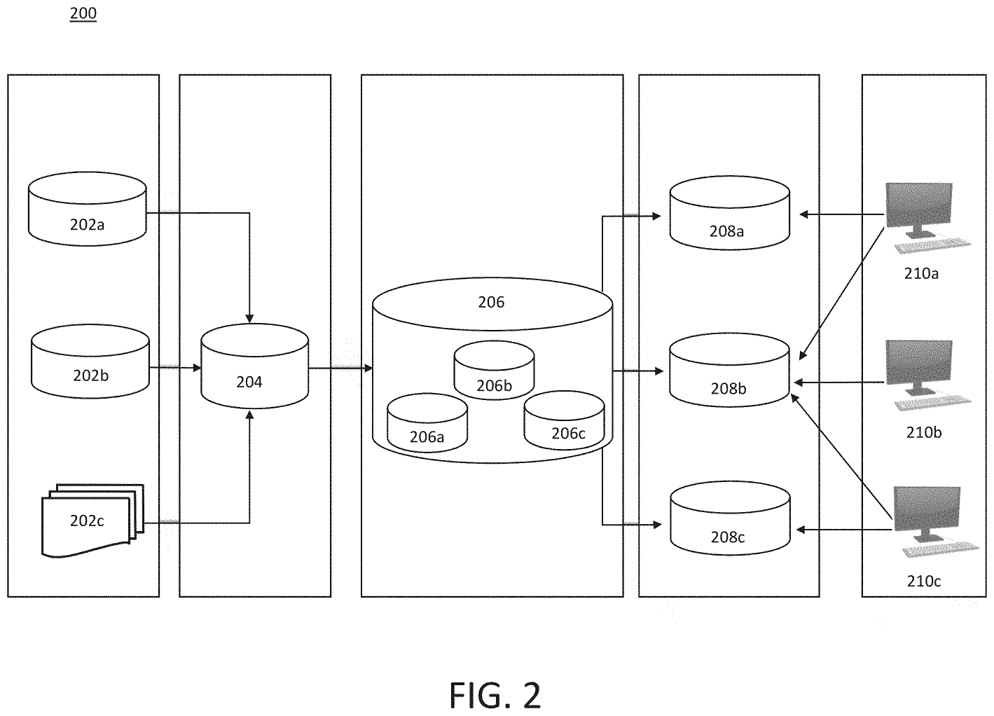

FIG. 2 illustrates an exemplary diagram of an ETL data pipeline 200 according to various embodiments of the present disclosure. In various embodiments, ETL data pipeline 200 includes one or more data sources 202a, 202b, 202c. The data sources 202a, 202b, 202c may include structured and/or unstructured data. In various embodiments, ETL data pipeline 200 may include operational systems 202a and 202b and flat files 202c. In various embodiments, ETL data pipeline 200 may include a data staging area 204, such as, for example, a data reservoir, where data is aggregated from the data sources 202a, 202b, 202c into a single database.

In various embodiments, ETL data pipeline 200 includes a data warehouse 206 configured to store transformed data after ETL processing, as described above. In various embodiments, the data warehouse 206 may include database subsystems 206a, 206b, and 206c. In various embodiments, database subsystem 206a may include summary data. In various embodiments, database subsystem 206b may include meta data. In various embodiments, database subsystem 206c may include raw data.

In various embodiments, ETL data pipeline 200 includes one or more datamarts 208a, 208b, and 208c configured to provide access to a specific subset of data in the data warehouse for access to, e.g., a specific business group or external customer, as described above. For example, datamart 208a may include processed data specific to a purchasing department. In another example, datamart 208b may include processed data specific to a sales department. In another example, datamart 208c may include processed data specific to an inventory department. In the healthcare context, for example, datamarts 208a, 208b, and 208c may include data specific to healthcare providers, private health insurers, and government payers, respectively. In various embodiments, one or more users 210a, 210b, and 210c may access one or more of the datamarts 208a, 208b, and 208c.

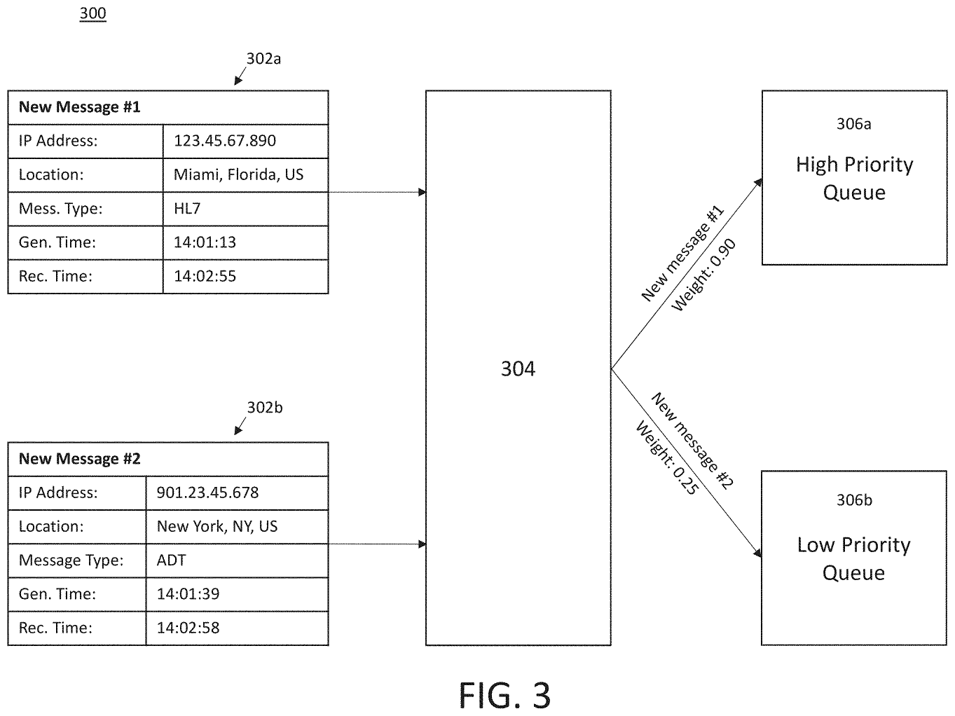

FIG. 3 illustrates an exemplary system 300 for prioritizing healthcare resources in an ETL system according to various embodiments of the present disclosure. In particular, a first new data message 302a having a first timestamp is received by an ETL system 304 and a second new data message 302b having a second timestamp is received by the ETL system 304. The ETL system 304 may be substantially similar to the systems described above and may receive new data messages 302a, 302b from a data reservoir and/or directly from data sources.

In various embodiments, the ETL system 304 may apply a crowdsourced model, as described in more detail above, to each of the new incoming data messages 302a, 302b. In various embodiments, the ETL system 304 may apply a weight to each of the messages. For example, the weight of the first new data message 3020a may be higher than the weight of the second new data message 302b and, thus, the first new data message 302a may be assigned to a high priority processing queue 306a and the second new data message 302b may be assigned to a low priority processing queue 306b.

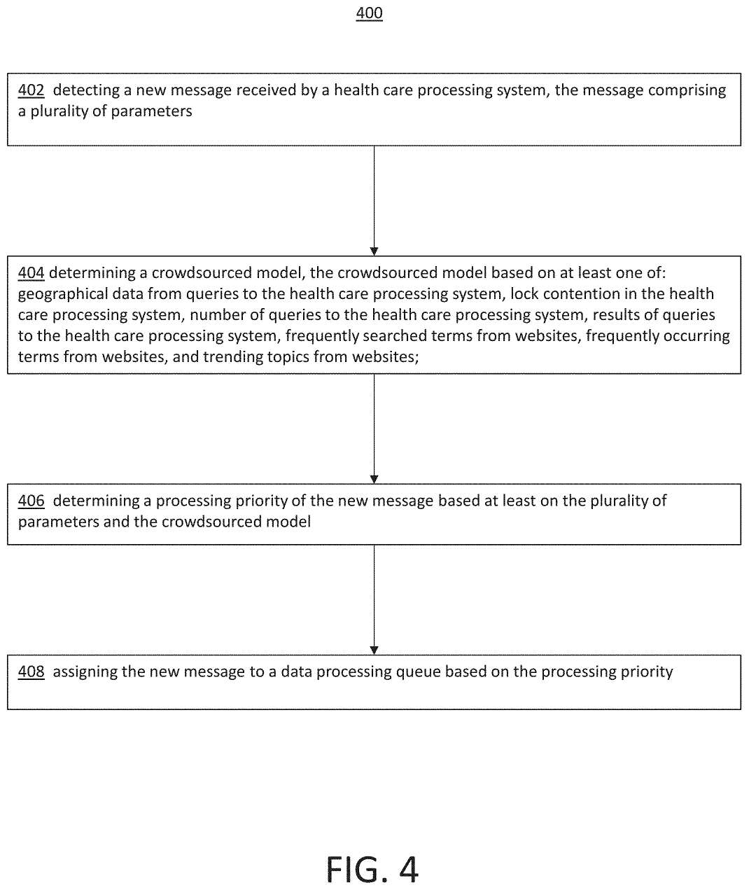

FIG. 4 illustrates a flow chart illustrating an exemplary method 400 for prioritizing processing resources for a health care processing system. At 402, a new message received by a health care processing system is detected. The message includes a plurality of parameters. At 404, a crowdsourced model is determined based on at least one of: geographical data from queries to the health care processing system, lock contention in the health care processing system, number of queries to the health care processing system, results of queries to the health care processing system, frequently searched terms from websites, frequently occurring terms from websites, and trending topics from websites. At 406, a processing priority is determined for the new message based at least on the plurality of parameters and the crowdsourced model. At 408, the new message is assigned to a data processing queue based on the processing priority.

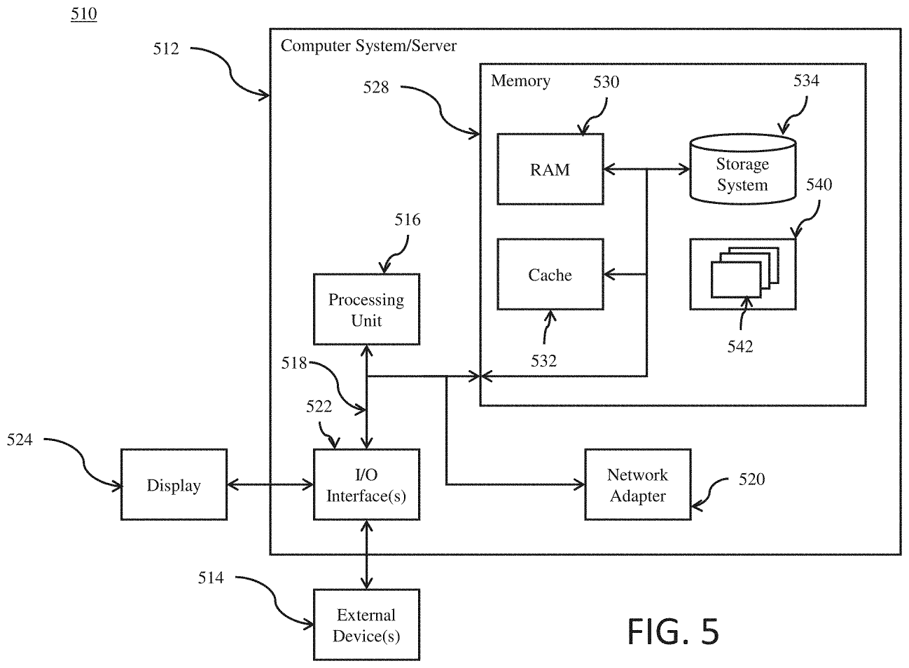

With reference to FIG. 5, a schematic of an example of a computing node is shown. Computing node 510 is only one example of a suitable computing node and is not intended to suggest any limitation as to the scope of use or functionality of embodiments of the invention described herein. Regardless, computing node 510 is capable of being implemented and/or performing any of the functionality set forth hereinabove.

In computing node 510 there is a computer system/server 512, which is operational with numerous other general purpose or special purpose computing system environments or configurations. Examples of well-known computing systems, environments, and/or configurations that may be suitable for use with computer system/server 512 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 512 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 512 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 512 in computing node 510 is shown in the form of a general-purpose computing device. The components of computer system/server 512 may include, but are not limited to, one or more processors or processing units 516, a system memory 528, and a bus 518 that couples various system components including system memory 528 to processor 516.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 512 typically includes a variety of computer system readable media. Such media may be any available media that is accessible by computer system/server 512, and it includes both volatile and non-volatile media, removable and non-removable media.

System memory 528 can include computer system readable media in the form of volatile memory, such as random access memory (RAM) 530 and/or cache memory 532. Computer system/server 512 may further include other removable/non-removable, volatile/non-volatile computer system storage media. By way of example only, storage system 534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 518 by one or more data media interfaces. As will be further depicted and described below, memory 528 may include at least one program product having a set (e.g., at least one) of program modules that are configured to carry out the functions of embodiments of the invention.

Program/utility 540, having a set (at least one) of program modules 542, may be stored in memory 528 by way of example, and not limitation, as well as an operating system, one or more application programs, other program modules, and program data. Each of the operating system, one or more application programs, other program modules, and program data or some combination thereof, may include an implementation of a networking environment. Program modules 542 generally carry out the functions and/or methodologies of embodiments of the invention as described herein.

Computer system/server 512 may also communicate with one or more external devices 514 such as a keyboard, a pointing device, a display 524, etc.; one or more devices that enable a user to interact with computer system/server 512; and/or any devices (e.g., network card, modem, etc.) that enable computer system/server 512 to communicate with one or more other computing devices. Such communication can occur via Input/Output (I/O) interfaces 522. Still yet, computer system/server 512 can communicate with one or more networks such as a local area network (LAN), a general wide area network (WAN), and/or a public network (e.g., the Internet) via network adapter 520. As depicted, network adapter 520 communicates with the other components of computer system/server 512 via bus 518. It should be understood that although not shown, other hardware and/or software components could be used in conjunction with computer system/server 512. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, and/or a deep Q-network (e.g., deep QA).

Artificial neural networks (ANNs) are distributed computing systems, which consist of a number of neurons interconnected through connection points called synapses. Each synapse encodes the strength of the connection between the output of one neuron and the input of another. The output of each neuron is determined by the aggregate input received from other neurons that are connected to it. Thus, the output of a given neuron is based on the outputs of connected neurons from preceding layers and the strength of the connections as determined by the synaptic weights. An ANN is trained to solve a specific problem (e.g., pattern recognition) by adjusting the weights of the synapses such that a particular class of inputs produce a desired output.

Various algorithms may be used for this learning process. Certain algorithms may be suitable for specific tasks such as image recognition, speech recognition, or language processing. Training algorithms lead to a pattern of synaptic weights that, during the learning process, converges toward an optimal solution of the given problem. Backpropagation is one suitable algorithm for supervised learning, in which a known correct output is available during the learning process. The goal of such learning is to obtain a system that generalizes to data that were not available during training.

In general, during backpropagation, the output of the network is compared to the known correct output. An n error value is calculated for each of the neurons in the output layer. The error values are propagated backwards, starting from the output layer, to determine an error value associated with each neuron. The error values correspond to each neuron's contribution to the network output. The error values are then used to update the weights. By incremental correction in this way, the network output is adjusted to conform to the training data.

When applying backpropagation, an ANN rapidly attains a high accuracy on most of the examples in a training-set. The vast majority of training time is spent trying to further increase this test accuracy. During this time, a large number of the training data examples lead to little correction, since the system has already learned to recognize those examples. While in general, ANN performance tends to improve with the size of the data set, this can be explained by the fact that larger data-sets contain more borderline examples between the different classes on which the ANN is being trained.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
detecting a new message received by a health care processing system, the message comprising a plurality of parameters;
determining a crowdsourced model, the crowdsourced model based on at least one of:
geographical data from queries to the health care processing system,
lock contention in the health care processing system,
number of queries to the health care processing system,
results of queries to the health care processing system,
frequently searched terms from websites,
frequently occurring terms from websites, and
trending topics from web sites;
determining a processing priority of the new message based at least on the plurality of parameters and the crowdsourced model; and
assigning the new message to a data processing queue based on the processing priority.

2. The method of claim 1, wherein detecting the new message comprises detecting new data received at a data reservoir.

3. The method of claim 2, further comprising sending the new message to an Extract Transform Load (ETL) server from the data reservoir based at least on the data processing queue.

4. The method of claim 1, wherein the plurality of parameters comprises TCP/IP information, geographic information, message type, generation time, and reception time.

5. The method of claim 4, wherein the message type is selected from the group consisting of HL7, ORU, ADT, and FHIR.

6. The method of claim 1, wherein the health care processing system comprises an electronic health record (EHR) database.

7. The method of claim 1, wherein the crowdsourced model comprises an artificial neural network.

8. The method of claim 1, wherein the websites comprise healthcare websites, search engines, or social media websites.

9. The method of claim 1, wherein determining a processing priority of the new message comprises determining a weight between 0.0 and 1.0 for the new message.

10. The method of claim 1, wherein assigning the new message to the data processing queue comprises assigning a position ahead of a previously processed message in the processing queue.

11. The method of claim 1, wherein assigning the new message to the data processing queue comprises assigning a position behind of a previously processed message in the processing queue.

12. The method of claim 1, wherein assigning the new message to the data processing queue comprises sending the new message to a high priority data processing queue.

13. The method of claim 1, wherein assigning the new message to the data processing queue comprises sending the new message to a low priority data processing queue.

14. The method of claim 1, wherein the heath care processing system supports multi-tenant data restricting access based on privacy requirements.

15. A system comprising:
a computing node comprising a computer readable storage medium having program instructions embodied therewith, the program instructions executable by a processor of the computing node to cause the processor to perform a method comprising:
detecting a new message received by a health care processing system, the message comprising a plurality of parameters;
determining a crowdsourced model, the crowdsourced model based on at least one of:
geographical data from queries to the health care processing system,
lock contention in the health care processing system,
number of queries to the health care processing system,
results of queries to the health care processing system,
frequently searched terms from websites,
frequently occurring terms from websites, and
trending topics from websites;
determining a processing priority of the new message based at least on the plurality of parameters and the crowdsourced model; and
assigning the new message to a data processing queue based on the processing priority.

16. The system of claim 15, wherein detecting the new message comprises detecting new data received at a data reservoir.

17. The system of claim 16, further comprising sending the new message to an Extract Transform Load (ETL) server from the data reservoir based at least on the data processing queue.

18. The system of claim 15, wherein assigning the new message to the data processing queue comprises sending the new message to a high priority data processing queue.

19. The system of claim 15, wherein assigning the new message to the data processing queue comprises sending the new message to a low priority data processing queue.

20. A computer program product for prioritizing processing resources for a health care processing system, the computer program product comprising a computer readable storage medium having program instructions embodied therewith, the program instructions executable by a processor to cause the processor to perform a method comprising:
detecting a new message received by a health care processing system, the message comprising a plurality of parameters;
determining a crowdsourced model, the crowdsourced model based on at least one of:
geographical data from queries to the health care processing system,
lock contention in the health care processing system,
number of queries to the health care processing system,
results of queries to the health care processing system,
frequently searched terms from websites,
frequently occurring terms from websites, and
trending topics from websites;
determining a processing priority of the new message based at least on the plurality of parameters and the crowdsourced model; and
assigning the new message to a data processing queue based on the processing priority.
